(19) United States
(12) Patent Application Publication
(10) Pub. No.: US 2004/0112123 A1
(43) Pub. Date: Jun. 17, 2004

(54) Self-propelled sensor apparatus for in situ analysis of environmental parameters

(76) Inventors: David P. Fries, St. Petersburg, FL (US); George Steimle, St. Petersburg, FL (US); Heather A. Broadbent, St. Petersburg, FL (US)

Correspondence Address: ALLEN, DYER, DOPPELT, MILBRATH & GILCHRIST, PA, P.O. BOX 3791, ORLANDO, FL, 32802-3791, US

(21) Appl. No.: 10/319,683

(22) Filed: Dec. 13, 2002

(51) Int. Cl. G01N 1/14; G01N 33/18; G01N 33/00

(52) U.S. Cl. 73/64.56; 73/31.02; 73/31.07; 73/863.23

(57) ABSTRACT

An analytical system is provided which uses the kinetic energy of motion of the entire system to drive a fluid under analysis through the system. This is accomplished by use of a propulsion system which is attached to the analytical portion of the system. Various sensor detection systems may be used to analyze the sample collected within the confines of a sample isolation and concentration module contained within the system. Trace quantities of suspect materials may be detected or monitored by use of the instant system.

### RELATED APPLICATIONS

This application is a Continuation-In-Part of application Ser. No. 09/xxx,xxx, filed mmm ddd, 2002, now U.S. Pat. No.

### STATEMENT OF GOVERNMENT INTEREST

The work that led to this invention has been supported in part by a grant from the Department of the Navy, Grant Number ONR -N0014-98-1-0848. Thus, the United States Government may have certain rights to this invention.

### FIELD OF THE INVENTION

The instant invention is directed to a fluid analysis method and apparatus, and particularly to such a method and apparatus for use in analysis of biological or chemical, particle or physical species contained in fluid milieus that include trace amounts. More specifically, the instant invention is concerned with an analytical apparatus that additionally contains a propulsion unit to convey the analytical unit through the analyte fluid to collect and/or detect desired materials.

### BACKGROUND OF THE INVENTION

In recent years the presence of contaminants in bodies of water both fresh and salt varieties has become an issue of both public and governmental interest. In addition, air quality with respect to pollution from industrial or bellicose activities deeply affects the daily lives of most of the world's population. With the changing political situation in the world as well as concern over contamination from industrial and agricultural activity, a new intense interest has developed in monitoring water and air sources for pollutants and trace quantities of materials. As technology progresses, it has become increasingly important to know immediately the content of a body of water or air, thus necessitating the development of new analytical systems to give precise information on the presence and/or quantities of microbial and chemical contaminants.

To date, the prior art method and devices have been concerned with “capturing” a sample for transportation to a laboratory for analysis and, in the case of trace quantities, concentration of the suspect species for that analysis. In addition, many of the prior art devices include sophisticated sensors and pumping apparatus which make the devices cumbersome as well as expensive to assemble and to maintain. Even though towed or tethered samplers have been known the art previously, their uses have been limited to physical characteristics and not monitoring of chemical or biological species.

U.S. Pat No. 3,537,316 to Stewart et al shows a towed underwater sampler having an internal cavity which houses sensor circuits. In this device, water is permitted to flow through the analysis chamber so that temperature and pressure may be evaluated. However, the sensors here are measuring physical parameters and not the chemical or biological content of the water passing through the sensor cavity. In fact, there is no actual sample reading made by the instrument only the desired parameters of temperature and pressure are evaluated and the actual sample is captured in a bottle for later analysis.

Another towed sensor system is disclosed in U.S. Pat. No. 4,713,967 to Overs et al. Again the sensors only are concerned with physical properties, these being temperature and water speed. In this patent, the speed and temperature are then equated to the presence of fish bait but no information is obtained about any compositional make-up of the environment or the nature of the fish bait itself.

Inner chambers in contaminant sensing devices for water analysis are described in the prior art as well. One example is U.S. Pat. No. 6,272,938 to Baghel et al who describes an inner chamber formed by a semi-permeable membrane in communication with an inner chamber containing a sensor to monitor contaminants in a tethered style apparatus. The water in this case diffuses through the membrane until a threshold is reached and then the diffusion is stopped. In this system, the quantity of contaminant is a function of diffusion time and thus is controlled by a unpredictable parameter.

U.S. Pat. No. 6,306,350 to Mereish et al describes a portable water sampling device which captures the sample in a chamber which is then removed and sent to a lab for analysis. Concentration here is a function of time since a timer is used to determine the sample collection period; in this patent a pump is also used to force the water being tested into the system and past the extraction membrane.

Similar devices which incorporate sampling chambers are described in U.S. Pat. No. 5,844,147 to Fiedler et al and U.S. Pat. No. 5,167,802 to Sandstrom et al. Again, the samples are collected and sent to a remote lab for analysis.

In addition to water environments, similar devices have been used in the atmosphere. Examples of these are U.S. Pat. No. 6,321,609 to Mengel et al and U.S. Pat. No. 6,354 135 to McGee et al. Again, these systems include suction devices or pumps to facilitate the flow of effluent through the monitoring apparatus.

It is readily apparent that a system for immediate analysis of contaminants in situ is needed to overcome the disadvantages of the prior art systems. It is also apparent that there is a need for a system that incorporates reliability and sensitivity in performing the necessary analyses which is low-cost and easy to maintain. It is, therefore, to the provision of such an instrument that the instant invention is directed.

### SUMMARY OF THE INVENTION

It is therefore an object of this invention to provide an improved apparatus and method of analyzing a fluid composition in situ or in an on-site situation.

It is another object of the invention to provide an improved sampling device which is capable of producing real time concentrations of trace contaminants without using remote testing facilities.

It is a further object of the invention to provide a device which is capable of measuring large volumes of fluids without having to use pumping systems to aid in the sampling process.

It is another object of the invention to provide a highly-sensitive contaminant assay system for fluid analysis without having to use expensive, highly-sensitive sensors.

It is a further object of the invention to provide a portable system capable of detecting, quantifying, or combinations thereof for use in various types of environments, including hostile.

It is another object of the invention to use the inertia of motion to provide the kinetics necessary to provide sample and effluent diffusion.

It is a further object of the invention to provide a system that includes a self-propulsion component to serve as the impelling force for both driving the analytical system through a fluid to be analyzed as well as driving the analyte through the analytical portion of the apparatus itself.

Still additional objects will become apparent as the invention is further described.

### BRIEF DESCRIPTION OF THE DRAWINGS

FIG. 1 is a block diagram of the complete analytical system of the invention.

FIG. 2 is a representation of one sampling chamber of the instant analytical system.

FIG. 3 represents another configuration of an optical sensor based sample chamber usable in the instant invention.

FIG. 4 is a further structure of an optical sensor type of detection system.

FIG. 5 is another embodiment showing a further geometry for the chamber of the instant invention.

FIG. 6 shows an overall schematic of the instant device including the propellant portion attached to the analytical portion.

### DETAILED DESCRIPTION OF THE PREFERRED EMBODIMENTS

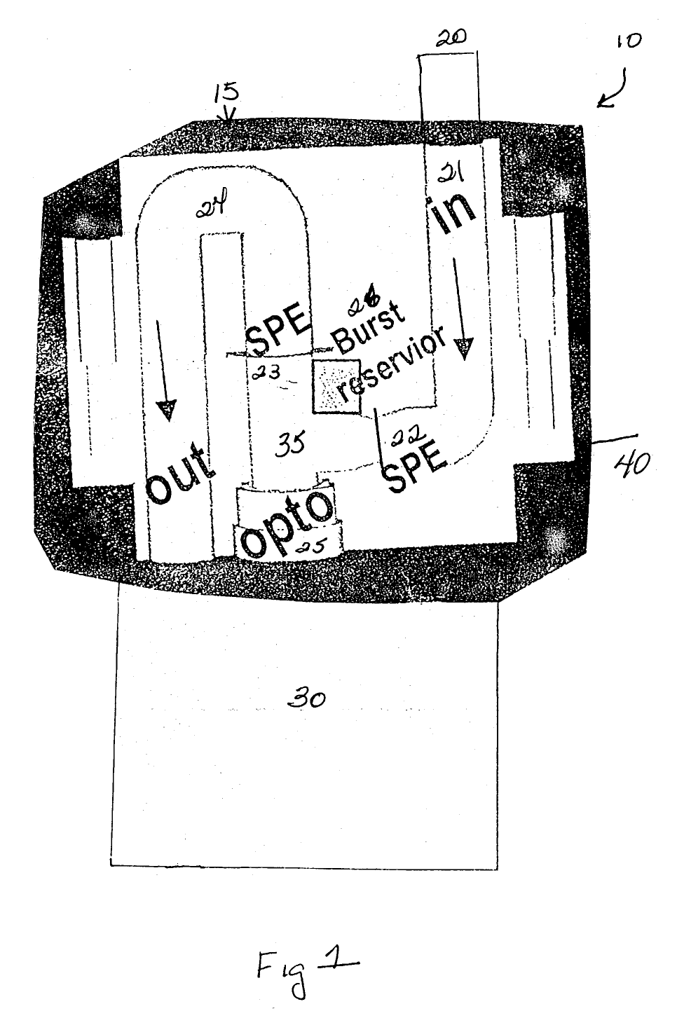

Referring now the FIG. 1, the analytical system 10 of the invention comprises a detection portion 15 connected to a support system 30. In the preferred embodiment, the two sections are encased in a housing means (not shown) which may be any suitable housing means as known to those of ordinary skill in the art for the environment of use. The two sections 15 and 30 may be detachably connected, a single unit, or arranged so that reuse of desired components may be performed. Any desired geometry for the overall system may be chosen by one of ordinary skill in the art, FIG. 1 represents only one preferred embodiment.

The detection portion 15 comprises a fluid intake means 21 for ingress of the analyte fluid. This intake means 21 may be co-extensive with the housing means, protrude therefrom or be recessed within the interior confines of the housing means. A pre-extractor means 20 may be also present at the proximal end of the fluid intake means 21 if desired to separate deleterious material from entering the detection portion 15. The fluid intake means 21 as well as the fluid outflow means 24 may be formed of any suitable material as known to those of ordinary skill in the art. In the preferred embodiment, any suitable plastic material which is non-porous and inert to the environment is the preferred material.

Located at the distal end of the fluid intake means 21 is a first separator means 22 which serves to block unwanted material from entering the analysis chamber 35. This separator means may be any suitable separator such as a filter, screening material, or in the preferred embodiment, a semi-permeable membrane. This first separator means 22 is chosen for the milieu of use and for optimizing the effectiveness of performing a concentrating and screening function, these systems being well-known to those of ordinary skill in the art.

A second separator means 23 is located in fluid communication with the first separator means 22 with the intermediate portion of the fluid pathway defining analysis chamber 35. The second separator means 23 is chosen to prevent the analyte of interest from exiting the analysis chamber 35 and is chosen of a material again suited for this purpose. In addition, both the first and second separator means may have coatings applied to them to assist in the detection of the analyte, such as, reflectance coatings applied to enhance optical characteristics of the system. The fluid of interest exits the analytical system 10 via fluid outlet means 24.

The analysis chamber 35 by virtue of separator means 23 also functions as a concentration means. Thus the material of interest is trapped within the confines of the analysis chamber 35 so that the sensor or other suitable sensing means 25 is able to respond to its presence. The sensing means 25 may be designed to respond to a threshold value or may be chosen to actually quantify the analyte contained in the analysis chamber 35. In addition, the sensing means 25 may be constructed to react to a plurality of analytes, thus being a multi-sensor type of device.

In addition, an optional burst reservoir 26 may be included in the structure of the detection portion 15, so that a chemical enhancement means may be introduced into the analysis chamber 35 to aid the sensing means 25 in performance of its task. Again, if a plurality of analyses are performed, this burst reservoir 26 may actually be compartmentalized and serve to introduce a plurality of enhancement means.

The support system 30 comprises the electronic components necessary to support the function of the sensing means 25. This includes power supplies, either battery or cable supplied as well as the support electronics necessary to run the sensors. In addition, any other necessary or desired support equipment may also be contained within this, these including, but not limited to, telemetry devices, GPS units, and data storage units.

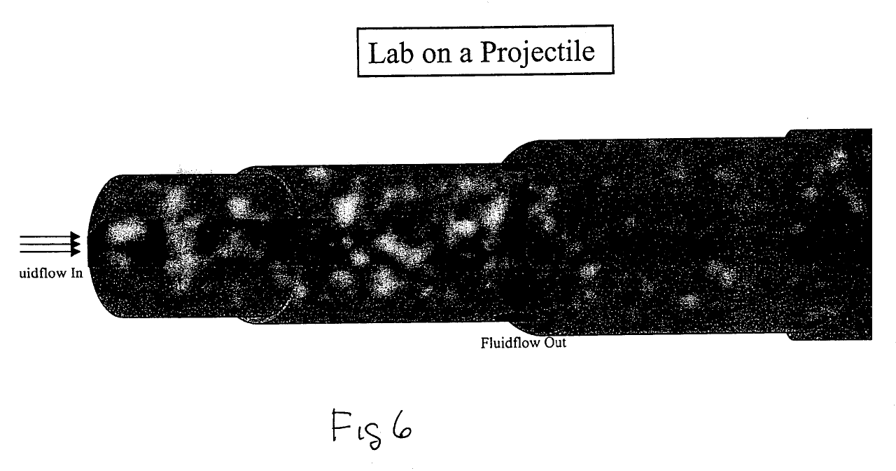

The analytical system 10 is connected to a propulsion means 80 as shown in FIG. 5. This may be any either an integral system to the overall device or a detachable propulsion section which may be even be replaceable if the overall system is intended to be reusable. Examples of propulsion system include, but are not limited to: bullets, artillery shells, torpedos, drop projectiles, fired projectiles, missiles, and other munition systems. In addition, telemetry systems may be included for relaying the desired data back to a monitoring station.

The propulsion system of the instant invention serves to not only transmit the analytical device to the location of interest, but also to provide the fluid flow within the system to effect the analytical functions. The sampling function may occur while the propulsion system is actively powering the device, or after the propulsion system is spent in a free-drift type of mode. Additional power sources may also be present for telemetry, GPS, electronic controls and other communication purposes. Further instrumentation may also include receivers, steering devices and other ground or ship communication devices, so that adjustments may be made to the flight path of the instrument after it is deployed. In addition, a second propulsion system may be incorporated into the device so that it may be transmitted after a period of time to a further location, such as a pick-up location. This may be an aerial type of device for overland applications or a flotation device for aqueous applications.

In a further embodiment, the propulsion means may be detachable so that the analytical system 10 may be released and gravity acts to propel it through the fluid medium. In this embodiment, telemetry may also be used to transmit the data or other results back to a monitoring station or the instrument may be retrieved. Even contemplated is the use of balloons, or kites with sampling taking place during ascent and travel, and if detachable cords are used, sampling may also occur during gravitational descent.

Because gravity or the motion of the conveyance means are used as the flow impelling means in the instant system, the need for the auxiliary pumping means of the prior art is obviated. This enables the instant device to be reduced in size and simplifies the power requirements of the system. In addition, the sample chamber 35 may be a micro-sized portion of the overall system, so that minute or trace amounts of analyte may be captured and detected.

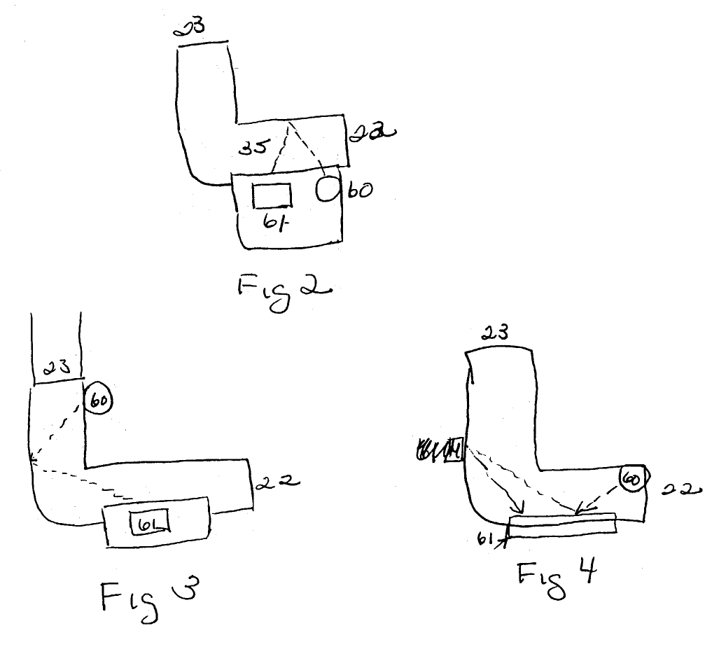

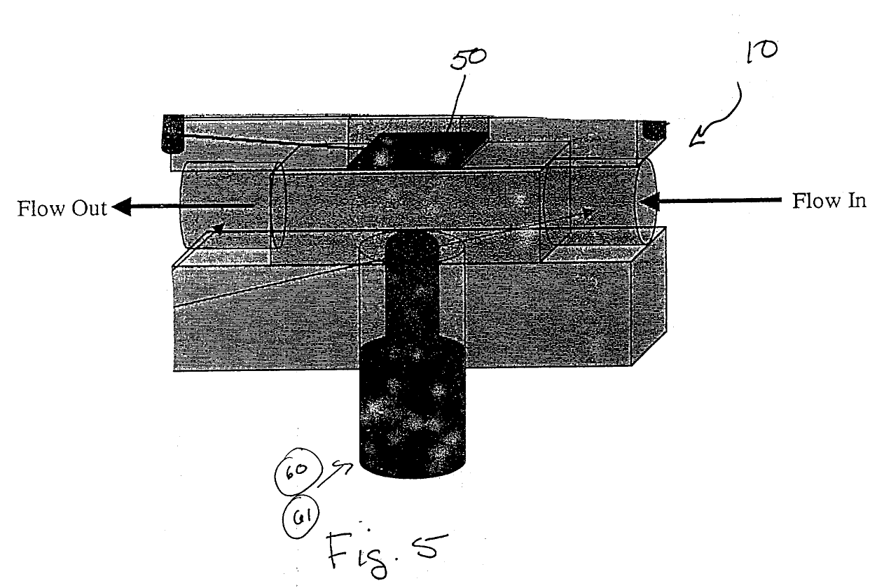

The sample chamber 35 may be constructed in any geometry necessary to enhance the performance of the chosen sensor and analyte system. Three possible geometries for an optical sensor type detection system are shown in FIGS. 2-5. In each of these systems a source 60 sends out a light beam through the sample chamber 35 to detection means 61. Other geometries are also available and are considered as design variations to one of ordinary skill in the art, including a linear arrangement as shown in FIG. 5.

In addition to a single detection system, it is contemplated that a flow splitting arrangement may also be incorporated so that multiple discreet detections may be made simultaneously. In addition, either one or both of the separator means 22 and 23 may be omitted depending on the sensor system used. Reagent systems which trap the analyte or assist in the detection may also be used. This type of format is shown in FIG. 5 in conjunction with a linear, non-membrane detection system. Here, a reagent trapping means 50 is used for isolation of the desired analyte.

In addition to optical sensor systems, various other type of sensor detection systems may be employed; these including, but not limited to, electrical, electrochemical, gravimetric, mass loading and ion or molecular and particle traps.

Various configurations of the sample chamber 35 to accommodate these types are systems are considered within the scope of knowledge to one of ordinary skill in the art.

In addition, a threshold type of sensor system may also be incorporated into the analytical system, with comparison to a pre-determined level being the output of choice.

Modification and variation can be made to the disclosed embodiment of the instant invention without departing from the scope of the invention as described. Those skilled in the art will appreciate that the applications of the present invention herein are varied, and that the invention is described in the preferred embodiment. Accordingly, additions and modifications can be made without departing from the principles of the invention. Particularly with respect to the claims it should be understood that changes may be made without departing from the essence of this invention. In this regard it is intended that such changes would still fall within the scope of the present invention. Therefore, this invention is not limited to the particular embodiments disclosed, but is intended to cover modifications within the spirit and scope of the present invention as defined in the appended claims.

What is claimed is:

1. An analytical apparatus for analysis of a component contained in a fluid medium comprising:
a. a fluid inlet means;
b. a fluid outlet means; said fluid inlet means connected to said fluid outlet means via a fluid conduit means defining a fluid pathway;
c. a sample confinement chamber; said sample confinement chamber located intermediate said fluid inlet means and said fluid outlet means in said fluid pathway and connected to said fluid conduit means;
d. a first separator means; said first separator means located at the proximal end of said sample confinement means in said fluid pathway and fluid conduit;
e. a second separator means; said second separator means located at the distal end of said sample confinement means in said fluid pathway and fluid conduit;
f. a detector means; said detector means located within said sample confinement means and in communication with said fluid pathway; and
g. a conveyance means; said conveyance means adapted to propel said analytical apparatus through the fluid medium.

2. The analytical apparatus of claim 1, wherein the conveyance means comprises a propulsion means connected to said analytical apparatus.

3. The apparatus of claim 2, wherein the propulsion means contains power support means for said analytical apparatus as well as power means for secondary communication equipment.

4. The apparatus of claim 2, wherein the propulsion means is renewable.

5. The apparatus of claim 2, wherein the propulsion means is detachable.

6. The apparatus of claim 1, wherein the apparatus additionally contains a power support means.

7. The apparatus of claim 1, wherein the apparatus additional contains a data transferring means.

8. The apparatus of claim 7, wherein the apparatus additionally contains a power support means.

9. The apparatus of claim 1, wherein the detector means comprises a sensor system.

10. The apparatus of claim 9, wherein the sensor system is selected from the group consisting of optical, electrochemical, electrical, gravimetric, mass loading, ion trap, molecular traps and particle traps and other sensor based systems.

11. The apparatus of claim 9, wherein the sensor system measures a threshold.

12. A method of analyzing a component of a fluidic system comprising:
a. providing a fluid inlet means;
b. providing a fluid outlet means; said fluid inlet and outlet means connected to each other via a fluid conduit means which defines a fluid pathway;
c. providing a sample containment means; said sample confinement means located intermediate said fluid inlet means and said fluid out means and in said fluid pathway and connected to said fluid conduit means;
d. providing a first separator means; said first separator means located at the proximal end of said sample confinement means in said fluid pathway and fluid conduit;
e. providing a second separator means; said second separator means located at the distal end of said sample confinement means in said fluid pathway and fluid conduit;
f. providing a detector means; said detector means located within said sample confinement means and in communication with said fluid pathway; and
g. providing a conveyance means; said conveyance means adapted to propel the fluid through the fluid conduit by movement of the entire analytical system.

13. The method of claim 12, wherein the conveyance means consists of a propulsion system.

14. The method of claim 13, wherein the propulsion system is renewable.

15. The method of claim 13, wherein the propulsion system detaches from the analytical system.

16. The method of claim 13, wherein a second power means is also supplied which transmits power to the analytical system and transmits data from the analytical system.

17. The method of claim 12, wherein data is transmitted from the analytical system to a remote location.

18. The method of claim 12, wherein the analytical system responds to a predetermined threshold as the result.
